(19) United States
(12) Patent Application Publication
(10) Pub. No.: US 2025/0368697 A1
(43) Pub. Date: Dec. 4, 2025

(54) MODIFIED PEPTIDE DERIVED FROM URECHIS UNICINCTUS IN PREPARATION OF DRUG FOR TREATING ALCOHOLIC LIVER DISEASE

(71) Applicant: Beijing Institute of Petrochemical Technology, Beijing (CN)

(72) Inventor: Kunlai Sun, Beijing (CN)

(21) Appl. No.: 19/215,626

(22) Filed: May 22, 2025

(30) Foreign Application Priority Data
May 30, 2024 (CN) 202410688753X

(51) Int. Cl. C07K 14/435; A61K 38/00; A61P 1/16

(52) U.S. Cl. CPC C07K 14/43536; A61P 1/16; A61K 38/00

(57) ABSTRACT

An application method of a modified peptide derived from Urechis unicinctus is to prepare a drug for treating alcoholic liver disease. The modified peptide derived from Urechis unicinctus is one or more selected from F2-1: IHVKF (SEQ ID NO: 1), F2-2: VHFKI (SEQ ID NO: 2), F4-1: GAWKP (SEQ ID NO: 3), F4-2: AGWKP (SEQ ID NO: 4), F5-1: GTLKP (SEQ ID NO: 5), F5-2: GTPKL (SEQ ID NO: 6), F7-1: IIVRM (SEQ ID NO: 7), and F7-2: IRMVI (SEQ ID NO: 8). The modified peptide derived from Urechis unicinctus can enhance the degradation of alcohol and boost the survival rate of cells with alcoholic liver disease, and also alleviate lipid metabolism and inflammation in mice, protecting liver from alcoholic liver disease. When applied in drug development for treating alcoholic liver disease, the application method offers low-risk, low-cost, and is simple to operate therapeutic options.

### TECHNICAL FIELD

The disclosure relates to the field of hepatopathy treatment technologies, and particularly to an application of a modified peptide derived from Urechis unicinctus in preparation of a drug for treating alcoholic liver disease (ALD).

### STATEMENT REGARDING SEQUENCE LISTING

The sequence listing associated with this application is provided in text format in lieu of a paper copy and is hereby incorporated by reference into the specification. The name of the XML file containing the sequence listing is 25024TBYX-USP1-SL.xml. The XML file is 7,983 bytes; is created on May 6, 2025; and is being submitted electronically via patent center.

### BACKGROUND

Alcohol, also referred to as ethanol, is a water-soluble small molecule that enters the blood via a stomach and a proximal small intestine, then distributes throughout a human body. The ethanol first reaches the portal vein, which is directly discharged into the liver. The liver is the primary organ exposed to alcohol, metabolizing 90% of the ethanol, while the remainder of the ethanol is excreted via urine, sweat, and respiration. ALD, a globally prevalent chronic hepatopathy caused by long-term or excessive alcohol consumption, with clinical manifestations including hepatic steatosis, hepatic fibrosis, alcoholic hepatitis, cirrhosis, and hepatocellular carcinoma. Excessive alcohol use is the third-leading risk factor for global disease and disability, directly causing 60 diseases and injuries and contributing to at least 200 other diseases and injuries. As the primary organ of ethanol metabolism, the liver has long been recognized as a major organ affected by alcohol. Ethanol and its bioactive metabolites, such as acetaldehyde-acetate, fatty acid ethyl esters, and ethanol-protein adducts, are considered hepatotoxins that direct or indirect exert toxic effects on the liver. It is estimated that alcohol use causes approximately 2.5 million deaths annually, with the majority attributed to ALD. Since 2011, the number of patients with hepatitis B and hepatitis C has decreased, while the number of patients with alcoholic fatty liver disease (AFLD) have increased annually. Like other end-stage hepatopathy, liver transplantation is viewed as an ultimate treatment for ALD. However, it involves high risks, costs, and difficulty in donor matching, along with severe post-post-transplant immune rejection, and necessitates long-term use of immunosuppressive drugs. Thus, there is an urgent need to develop new ALD specific therapeutic agents.

### SUMMARY

Aiming to address the high risks, costs and difficulties in donor-recipient matching of liver transplantation for alcoholic and hepatic steatosis, as well as the strong postoperative immune rejection and the need for long-term immunosuppressive drug use, the disclosure provides an application of a modified peptide derived from Urechis unicinctus in preparation of a drug for treating alcoholic liver disease.

In an application method of a modified peptide derived from Urechis unicinctus in preparation of a drug for treating alcoholic liver disease, the modified peptide derived from Urechis unicinctus is selected from at least one selected from the group consisting of:
F2-1 with the amino acid sequence of SEQ ID NO: 1: IHVKF,
F2-2 with the amino acid sequence of SEQ ID NO: 2: VHFKI,
F4-1 with the amino acid sequence of SEQ ID NO: 3: GAWKP,
F4-2 with the amino acid sequence of SEQ ID NO: 4: AGWKP,
F5-1 with the amino acid sequence of SEQ ID NO: 5: GTLKP,
F5-2 with the amino acid sequence of SEQ ID NO: 6: GTPKL,
F7-1 with the amino acid sequence of SEQ ID NO: 7: IIVRM, and
F7-2 with the amino acid sequence of SEQ ID NO: 8: IRMVI;
where I represents isoleucine, H represents histidine, V represents valine, K represents lysine, F represents phenylalanine, G represents glycine, A represents alanine, W represents tryptophan, P represents proline, T represents threonine, L represents leucine, R represents arginine, and M represents methionine.

In an embodiment, the modified peptide derived from Urechis unicinctus is capable of activating alcohol dehydrogenase (ADH) enzyme, thereby enhancing a degradation efficiency of alcohol.

In an embodiment, the modified peptide derived from Urechis unicinctus is capable of increasing a survival rate of damaged liver cells.

In an embodiment, the modified peptide derived from Urechis unicinctus is capable of reducing lipid accumulation and hepatic steatosis in alcoholic liver cells.

In an embodiment, the modified peptide derived from Urechis unicinctus is capable of regulating a level of at least one selected from the group consisting of alanine transaminase (ALT), aspartate aminotransferase (AST), total cholesterol (TC), triglycerides (TG), low-density lipoprotein cholesterol (LDL-C), and high-density lipoprotein cholesterol (HDL-C) in the alcoholic liver cells.

In an embodiment, the modified peptide derived from Urechis unicinctus is capable of suppressing a pro-inflammatory cytokine in serum and reducing inflammatory responses in the alcoholic liver cells.

In an embodiment, the pro-inflammatory cytokines is at least one selected from the group consisting of cytochrome P450 2E1 (CYP2E1), interleukin-1β (IL-1β), and interleukin-6 (IL-6).

The disclosure further provides a drug or a drug combination for treating alcoholic liver damage, which includes the modified peptide derived from Urechis unicinctus mentioned above.

In an embodiment, the drug for treating alcoholic liver damage is the drug is in a form of one of tablets, coated tablets, dragee, pills, capsules, and sustained release tablets.

In an embodiment, the drug for treating alcoholic liver damage includes the modified peptide derived from Urechis unicinctus of F2-1, pirfenidone, curcumin, S-adenosyl-L-methionine (SAM-e), probiotic freeze-dried powder of Bifidobacterium, and coenzyme Q10 (ubiquinone).

In an embodiment, the drug for treating alcoholic liver damage is taken once daily with one capsule each time at night, and taking the drug at night is to avoid a peak period of alcohol metabolism.

In an embodiment, the drug for treating alcoholic liver damage includes the modified peptide derived from Urechis unicinctus of F2-1, silymarin, diammonium glycyrrhizinate, N-acetylcysteine (NAC), DL-α-tocopherol (vitamin E), and selenium yeast.

In an embodiment, the drug for treating alcoholic liver damage is taken once daily with one sustained release tablet each time before meals.

The modified peptide derived from Urechis unicinctus provided by the disclosure can enhance the alcohol degradation efficiency and survival rate of the alcoholic liver cells, alleviate lipid metabolism disorders and reduce inflammatory responses in mice, thereby protecting the mice from alcoholic liver disease. When applied in a preparation of drugs for treating alcoholic liver disease, it offers low therapeutic risks, low costs, and a simple and easy-to-operate method.

### BRIEF DESCRIPTION OF DRAWINGS

FIG. 1 illustrates a screening of peptides with a highest rate of alcohol metabolism based on an activation rate of alcohol dehydrogenase (ADH).

FIG. 2 illustrates effects of alcohol concentration on cell viability.

FIG. 3 illustrates protective effects of a modified peptide derived from Urechis unicinctus of F2-1 on alcoholic damage in human hepatocellular carcinomas (HepG2) cells at different concentrations.

FIG. 4 illustrates an effect of the modified peptide derived from Urechis unicinctus of F2-1 on a content of alanine aminotransferase (ALT).

FIG. 5 illustrates an effect of the modified peptide derived from Urechis unicinctus of F2-1 on a content of aspartate aminotransferase (AST).

FIG. 6 illustrates an effect of the modified peptide derived from Urechis unicinctus of F2-1 on a content of total cholesterol (TC).

FIG. 7 illustrates an effect of the modified peptide derived from Urechis unicinctus of F2-1 on a content of triglycerides (TG).

FIG. 8 illustrates an effect of the modified peptide derived from Urechis unicinctus of F2-1 on a content of high-density lipoprotein cholesterol (HDL-C).

FIG. 9 illustrates an effect of the modified peptide derived from Urechis unicinctus of F2-1 on a content of low-density lipoprotein cholesterol (LDL-C).

FIG. 10 illustrates an effect of the modified peptide derived from Urechis unicinctus of F2-1 on activity of Cytochrome P450 2E1 (CYP2E1).

FIG. 11 illustrates an effect of the modified peptide derived from Urechis unicinctus of F2-1 on activity of interleukin-1β (IL-1β).

FIG. 12 illustrates an effect of the modified peptide derived from Urechis unicinctus of F2-1 on activity of interleukin-6 (IL-6).

FIG. 13 illustrates an effect of the modified peptide derived from Urechis unicinctus of F2-1 on liver tissues of national institute on alcohol abuse and alcoholism (NIAAA) mice (H&E staining, with a 100-magnification).

FIG. 14 illustrates an effect of the modified peptide derived from Urechis unicinctus of F2-1 on kidney tissues of the NIAAA mice (H&E staining, with the 100-magnification).

FIG. 15 illustrates the effect of the modified peptide derived from Urechis unicinctus of F2-1 on the content of ALT.

FIG. 16 illustrates the effect of the modified peptide derived from Urechis unicinctus of F2-1 on the content of AST.

FIG. 17 illustrates the effect of the modified peptide derived from Urechis unicinctus of F2-1 on the content of TC.

FIG. 18 illustrates the effect of the modified peptide derived from Urechis unicinctus of F2-1 on the content of TG.

FIG. 19 illustrates the effect of the modified peptide derived from Urechis unicinctus of F2-1 on the content of HDL-C.

FIG. 20 illustrates the effect of the modified peptide derived from Urechis unicinctus of F2-1 on the content of LDL-C.

FIG. 21 illustrates the effect of the modified peptide derived from Urechis unicinctus of F2-1 on the activity of CYP2E1.

FIG. 22 illustrates an effect of the modified peptide derived from Urechis unicinctus of F2-1 on a content of tumor necrosis factor-α (TNF-α).

FIG. 23 illustrates the effect of the modified peptide derived from Urechis unicinctus of F2-1 on a content of IL-6.

FIG. 24 illustrates an effect of the modified peptide derived from Urechis unicinctus of F2-1 on a content of inducible nitric oxide synthase (iNOS).

### DETAILED DESCRIPTION OF EMBODIMENTS

In order to make the above objectives, features, and advantages of the disclosure more apparent and understandable, the specific embodiments of the disclosure will be described in detail below in conjunction with the attached drawings, but it cannot be understood as limiting the scope of the disclosure.

### Embodiment 1

### Study on Protective Effect of Modified Peptide Derived From Urechis unicinctus on Alcoholic Liver Damage in Human Hepatocellular Carcinomas (HepG2) Cells

### 1.1 Experimental Materials and Instruments

### 1.1.1 Experimental Cells

The HepG2 cells are obtained from the Shanghai Cell Bank of the Chinese Academy of Sciences.

### 1.1.2 Experimental Drugs

Modified peptide derived from Urechis unicinctus is synthesized by Shanghai Chutai Biotechnology Co., Ltd., with a purity of ≥95%.

TABLE 1-1
Amino acid sequence (N-terminus to C-terminus,
i.e., N--C) and molecular weight of the peptide
derived from Urechis unicinctus

| | Molecular weight | Amino acid |
|---|---|---|
| Name | (in daltons, da) | sequence (N--C) |
| F2-1 | 642.78 | SEQ ID NO: 1: IHVKF |
| F2-2 | 642.78 | SEQ ID NO: 2: VHFKI |
| F4-1 | 557.64 | SEQ ID NO: 3: GAWKP |
| F4-2 | 557.64 | SEQ ID NO: 4: AGWKP |
| F5-1 | 514.61 | SEQ ID NO: 5: GTLKP |
| F5-2 | 514.61 | SEQ ID NO: 6: GTPKL |
| F7-1 | 630.84 | SEQ ID NO: 7: IIVRM |
| F7-2 | 630.84 | SEQ ID NO: 8: IRMVI |

### 1.1.3 Experimental Reagents

TABLE 1-2
Experimental reagents

| Reagents | Manufacturers |
|---|---|
| Anhydrous ethanol, 95% ethanol | Sinopharm Chemical Reagent |
| | Co., Ltd. |
| Procell HepG2 specific culture | Wuhan Procell Life Science & |
| medium | Technology Co., Ltd. |
| 0.25% Trypsin-EDTA | Beijing Solarbio Technology |
| | Co., Ltd. |
| Sodium penicillin/streptomycin | Beijing Solarbio Technology |
| sulfate double antibody solution | Co., Ltd. |
| (P/S solution) | |
| Triglyceride (TG) assay kit | Nanjing Jiancheng Bioengineering |
| | Institute |
| Cell Counting Kit-8 (CCK-8 assay | Beijing Solarbio Technology |
| kit) | Co., Ltd. |
| Total cholesterol (T-CHO) assay | Nanjing Jiancheng Bioengineering |
| kit | Institute |
| Silymarin | Nanjing Jiancheng Bioengineering |
| | Institute |
| Alanine aminotransferase (ALT) | Tianshilife Pharmaceutical Group |
| assay kit | Co., Ltd. |
| Aspartate aminotransferase (AST) | Nanjing Jiancheng Bioengineering |
| assay kit | Institute |
| High-density lipoprotein cholesterol | Nanjing Jiancheng Bioengineering |
| (HDL-C) assay kit | Institute |
| Low-density lipoprotein cholesterol | Nanjing Jiancheng Bioengineering |
| (LDL-C) assay kit | Institute |
| Interleukin 1β (IL-1β) ELISA | Hangzhou Leyi Biotechnology |
| assay kit | Co., Ltd. |
| Interleukin 6 (IL-6) ELISA assay | Hangzhou Leyi Biotechnology |
| kit | Co., Ltd. |
| Cytochrome P450 2E1 (CYP2E1) | Hangzhou Leyi Biotechnology |
| ELISA assay kit | Co., Ltd. |
| RNAeasyTM Animal RNA | Biyoxy Biotechnology Co., Ltd. |
| Extraction Kit | |
| ABScript III RT Master Mix for | US ABclonal Biological Co., Ltd. |
| qPCR with gDNA Remover | |
| (RK20429) | |
| 2X Universal SYBR Green Fast | US ABclonal Biological Co., Ltd. |
| qPCR Mix (RK21203) | |

### 1.1.4 Main Instruments

TABLE 1-3
Main instruments

| Instruments | Manufacturers |
|---|---|
| −80° C. Thermo U/L Freezer | Thermo Fisher Scientific (China) |
| | Co., Ltd. |
| TGL-16G High-Speed Centrifuge | Shanghai Anting Scientific |
| | Instrument Factory |
| TI-S Inverted Fluorescence | Nikon Corporation (Japan) |
| Microscope | |
| MSA225S Analytical Balance | Sartorius Scientific Instruments |
| | (China) Co., Ltd. |
| Multisjan F Microplate Reader | Bio-Rad Laboratories (Shanghai) |
| | Co., Ltd. |
| Cell Incubator | Thermo Fisher Scientific (China) |
| | Co., Ltd. |
| PCR Thermal Cycler (T100) | Bio-Rad Laboratories, Inc. (USA) |
| Real-Time PCR System (CFX | Bio-Rad Laboratories, Inc. (USA) |
| Connect) | |
| Ultra-Micro Protein Nucleic | BioDroppLite+ |
| Acid Analyzer | |
| High-Speed Refrigerated | Dalong Xingchuang Laboratory |
| Centrifuge | Instruments (Beijing) Co., Ltd. |
| DF-101S Constant Temperature | Zhengzhou Ketai Laboratory |
| Heating Magnetic Stirrer | Equipment Co., Ltd. |

### 1.2 Experimental Methods

### 1.2.1 Alcohol Dehydrogenase (ADH) Activation Rate Test

ADH is considered a rate-limiting step in alcohol metabolism within the body, and there is a strong correlation between an activation rate of ADH and a rate of alcohol metabolism in the body. Therefore, the activation rate of ADH in vitro is used to reflect an ability of samples to metabolize alcohol in vivo. Eight modified peptides derived from Urechis unicinctus listed in Table 1-1 are tested using an improved kit, with a detection method based on the following equation:

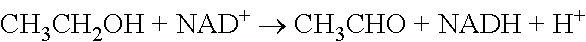

Nicotinamide adenine dinucleotide (NADH) has a maximum absorption peak at 340 nanometers (nm). A specific procedure is as follows: 50 microliters (μL) of a sample solution (0.1 milligrams per milliliter, abbreviated as mg/mL) is mixed with 150 μL of a detection reagent (containing buffer, NAD, and ethanol). After equilibrating at 37° C. for 5 minutes, 50 μL of ADH (0.2 units per milliliter, abbreviated as U/mL) is added to initiate a reaction. An absorbance at 340 nm is measured using a microplate reader, with readings taken every 15 seconds for a total of 10 minutes. Distilled water is used as a negative control in place of the sample solution. The reaction kinetic curve is fitted, and a first derivative of the curve at 0 minutes is obtained, which represents an initial reaction rate. The initial reaction rate can characterize relative enzyme activity of the ADH. The initial reaction rate of the sample solution is recorded as V_{S}, and that of the negative control as V₀. The ADH activation rate of the sample solution can be calculated using the following equation:

$$ADH activation rate ( \% ) : ( {V}_{S} - {V}_{0} ) / {V}_{0} \times 100 \%$$

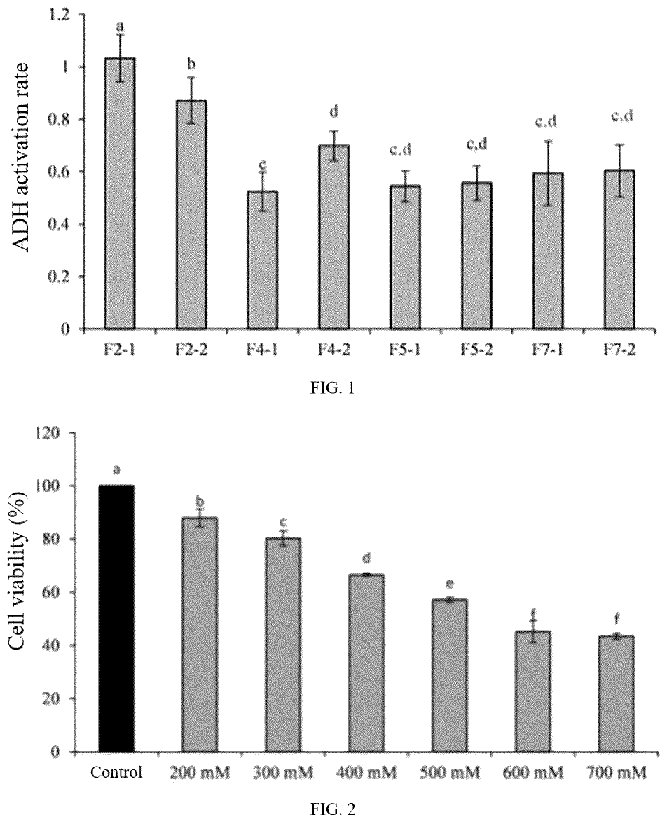

Test results are shown in FIG. 1. Note: No significant differences are observed between samples labeled with the same letters (a-d) (probability value >0.05, abbreviated as P>0.05).

1.2.2 Protective Effect of Modified Peptide Derived From Urechis unicinctus on Alcoholic Damaged Liver Cells

### 1.2.2.1 Cultivation of HepG2 Cells

Cell thawing: cryopreserved cells are taken out from an ultra-low temperature freezer and quickly thawed in a 37° C. water bath. The cells are then transferred into a 10 ml Eppendorf (EP) tube containing 5 mL of complete culture medium to mitigate the damage caused by dimethyl sulfoxide (DMSO). The EP tube is placed in a low-speed centrifuge and spun at 1000 revolutions per minute (rpm) for 5 minutes. The supernatant is discarded. Procell's HepG2-specific culture medium is added, and the cells are gently pipetted to form a single-cell suspension. The cells are then incubated in a 37° C., 5% CO₂ incubator for 12 hours before the medium is changed. The cells are continued to be cultured until they reach a density of 90%, at which point they are digested and passaged using 0.25% trypsin containing ethylene diamine tetraacetic acid (EDTA). The cells within the first two passages after thawing are not suitable for experiments. Experiments are conducted starting from a third passage. Aseptic techniques are strictly observed throughout the process.

### 1.2.2.2 Establishment of Alcoholic Damaged HepG2 Cell Model

When a cell density of the cells reached 90%, the cells are digested and passaged to form a single-cell suspension. The cell concentration is determined using a hemocytometer, and the suspension is adjusted to 8×10⁴ cells per well. A volume of 180 μL of the cell suspension is added to each well of a 96-well plate. The 96-well plate is then incubated in a 37° C., 5% CO₂ cell incubator for 24 hours. The cells are subjected to alcoholic damage for 24 hours using gradient concentrations of anhydrous ethanol at 200 micromoles per liter (μM), 300 μM, 400 M, 500 μM, 600 μM, and 700 μM (final concentrations). A blank group received no addition. All solutions in the 96-well plate are aspirated, and 100 μL of a 10% CCK-8 solution is added to each well. After incubation for 1 hour, the absorbance is measured at 450 nanometers (nm) using a microplate reader. The cell viability is calculated using the following formula:

$$Cell viability ( \% ) = ( {OD}_{experimental group} / {OD}_{blank group} ) \times 100 \%$$

The results are shown in FIG. 2. No significant differences are observed between groups labeled with the same letters (a-f) (P>0.05).

1.2.2.3 Protective Effect of Concentration of Modified Peptide Derived From Urechis unicinctus on HepG2 Cell Damage

HepG2 cells are collected, digested, centrifuged, and resuspended. The HepG2cells are then seeded into a 96-well plate at a density of 8×104 cells per well, with 160 μL of cell suspension added to each well. After incubating the plate in a 37° C., 5% CO₂ incubator for 24 hours, 20 μL of distilled water is added to a blank well as the blank group. Referring to Section 1.2.2.2 and the preparation of blank group cell sample, an anhydrous ethanol-induced damage model is established. The HepG2 cells are damaged with 500 mM anhydrous ethanol for 24 hours. The model group received 20 μL of distilled water, while the positive control wells received 20 μL of N-acetylcysteine (NAC) at a final concentration of 3 mM. The treatment wells receive 20 μL of the modified peptide derived from Urechis unicinctus of F2-1 at final concentrations of 200 μM, 300 μM, and 400 μM (dissolved in water). The 96-well plate is then incubated in a 37° C., 5% CO₂ incubator for 24 hours after adding the modeling solution. After incubation, all solutions in the 96-well plates are aspirated, and 100 μL of a 10% CCK-8 solution is added to each well. The absorbance is measured at 450 nm using a microplate reader after 1 hour of incubation. The cell viability is calculated using the following formula:

$$Cell viability ( \% ) = ( {OD}_{experimental group} / {OD}_{blank group} ) \times 100 \%$$

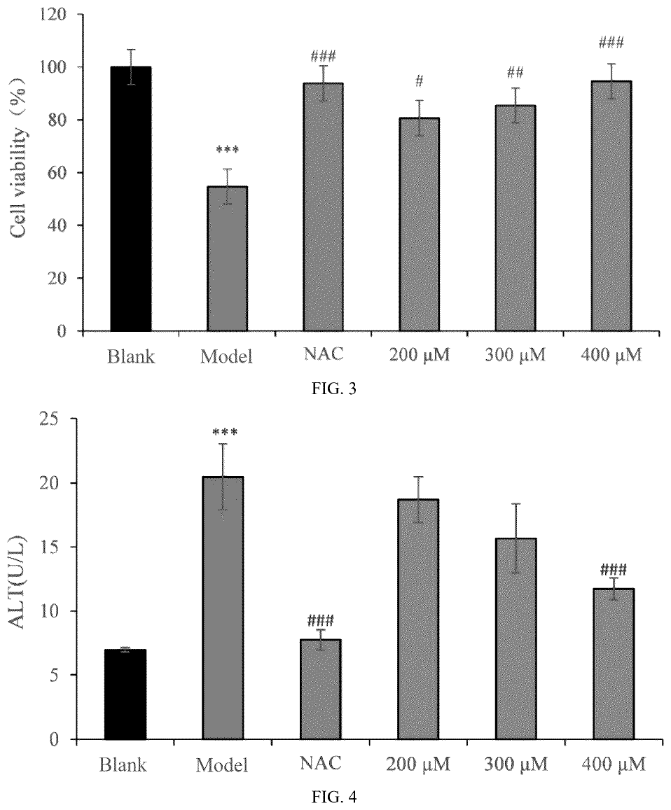

The results are shown in FIG. 3. Compared with the blank group: ***P<0.001, **P<0.01, *P<0.05; compared with the model group: ^{###}P<0.001, ^{##}P<0.01, ^{#}P <0.05.

1.2.3 Functional Protective Effect of Modified Peptide Derived From Urechis unicinctus on Alcoholic Damaged HepG2 Cell

### 1.2.3.1 Preparation of Cell Samples

HepG2 cells are collected, digested, centrifuged, and resuspended. The HepG2 cells are then seeded into a 6-well plate at a density of 1×10⁶ cells per well, with 1.6 mL of cell suspension added to each well. After incubating the plate in a 37° C., 5% CO₂ incubator for 24 hours, 200 μL of distilled water is added to the blank wells as the blank group. Referring to Section 1.2.2.2 and the preparation of blank group cell sample, an anhydrous ethanol-induced damage model is established. The HepG2 cells are damaged with 500 mM anhydrous ethanol for 24 hours. The model group receives 200 μL of distilled water, while the positive control wells receive 200 μL of NAC at a final concentration of 3 mM. The treatment wells receive 200 μL of the modified peptide derived from Urechis unicinctus of F2-1 at final concentrations of 200 μM, 300 μM, and 400 μM (dissolved in water). After adding the modeling solution, the plate is incubated in a 37° C., 5% CO₂ incubator for an additional 24 hours. The 6-well plate is then taken out and placed on ice. All liquids in the wells are discarded, and the cells are washed three times with pre-chilled phosphate buffer solution (PBS). High-efficiency radio immunoprecipitation assay (RIPA) lysis buffer is added, and the HepG2 cells are lysed on ice for 30 minutes. The lysates are then centrifuged at 12,000 rpm for 10 minutes at low temperature, and the white precipitate is discarded.

### 1.2.3.2 Determination of Biochemical Indicators in Cells

Reagent kits are purchased from Nanjing Jiancheng Bioengineering Institute. An enzyme-linked immunosorbent assay (ELISA) plate reader is used to measure biochemical indicators such as alanine aminotransferase (ALT), aspartate aminotransferase (AST), triglycerides (TG), total cholesterol (TC), high-density lipoprotein cholesterol (HDL-C), and low-density lipoprotein cholesterol (LDL-C).

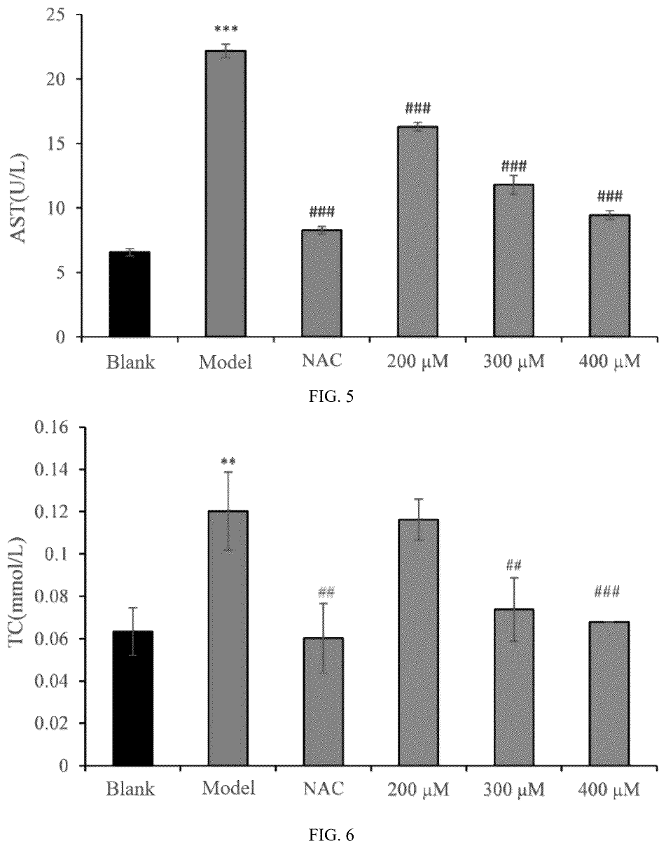

An effect of the modified peptide derived from Urechis unicinctus of F2-1 on ALT content is shown in FIG. 4. Compared with the blank group: ***P<0.001, **P<0.01, *P<0.05; compared with the model group: ^{###}P <0.001, ^{##}P <0.01, ^{#}P<0.05. The effect of the modified peptide derived from Urechis unicinctus of F2-1 on the AST content is shown in FIG. 5. Compared with the blank group: ***P<0.001, **P<0.01, *P<0.05; compared with the model group: ^{###}P <0.001, ^{##}P<0.01, ^{#}P<0.05.

Protective effects of the modified peptide derived from Urechis unicinctus of F2-1 on alcoholic damaged liver at different concentrations are evaluated based on levels of cholesterol (T-CHO) and TG in HepG2 cells. The two indicators are important for assessing lipid abnormalities. The effect of the modified peptide derived from Urechis unicinctus of F2-1 on TC content is shown in FIG. 6. Compared with the blank group: ***P<0.001, **P<0.01, *P<0.05; compared with the model group: ^{###}P<0.001, ^{##}P<0.01, ^{#}P<0.05.

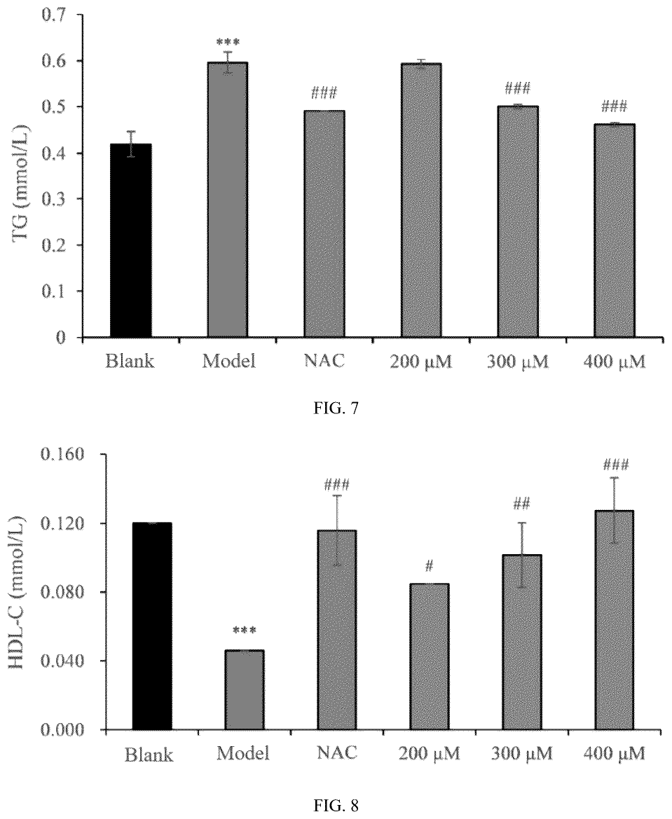

The effect of the modified peptide derived from Urechis unicinctus of F2-1 on TG content is shown in FIG. 7. Compared with the blank group: ***P<0.001, **P<0.01, *P<0.05; compared with the model group: ^{###}P<0.001, ^{##}P<0.01, ^{#}P<0.05.

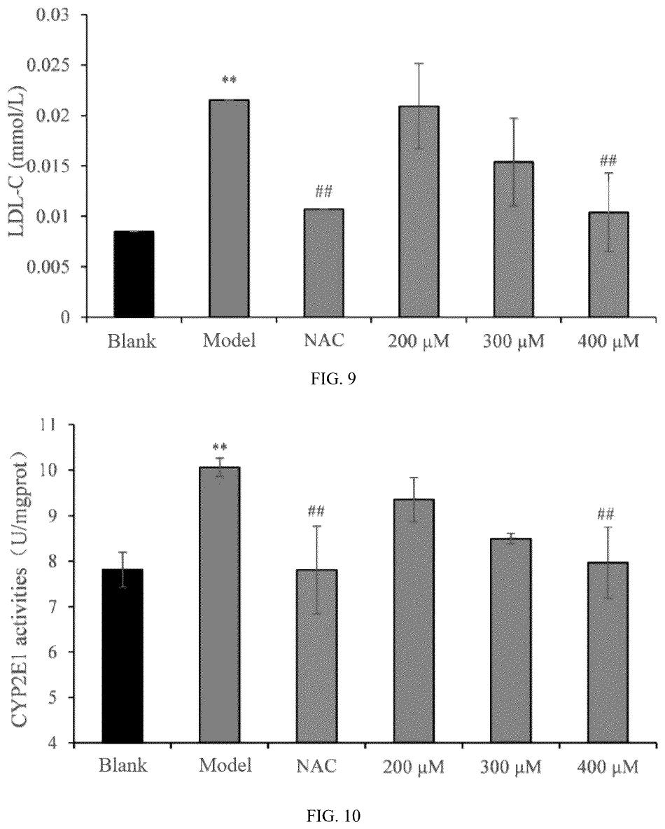

Protective effects of the modified peptide derived from Urechis unicinctus of F2-1 on alcoholic damaged liver at different concentrations are also evaluated based on levels of HDL-C and LDL-C in HepG2 cells. The two indicators are important for assessing lipid abnormalities. The effect of the modified peptide derived from Urechis unicinctus of F2-1 on HDL-C content is shown in FIG. 8. Compared with the blank group: ***P<0.001, **P<0.01, *P<0.05; compared with the model group: ^{###}P<0.001, ^{##}P<0.01, ^{#}P<0.05. The effect of the modified peptide derived from Urechis unicinctus of F2-1 on LDL-C content is shown in FIG. 9. Compared with the blank group: ***P<0.001, **P<0.01, *P<0.05; compared with the model group: ^{###}P<0.001, ^{##}P<0.01, ^{#}P<0.05.

### 1.2.3.3 Determination of Cellular ELISA Indicators

ELISA kits are purchased from Hangzhou Leyi Biotechnology Co., Ltd. An ELISA plate reader is used to measure indicators such as the interleukin-1β (IL-1β) ELISA kit, interleukin-6 (IL-6) ELISA kit, and Cytochrome P450 2E1 (CYP2E1) ELISA kit.

The protective effect of the modified peptide derived from Urechis unicinctus of F2-1 on HepG2 cells at different concentrations is evaluated based on levels of CYP2E1 in HepG2 cells. CYP2E1 is an enzyme found in the endoplasmic reticulum (ER) and hepatocyte mitochondria, which metabolizes alcohol into acetaldehyde in the presence of oxygen. Acetaldehyde directly upregulates the expression of sterol regulatory element-binding protein-1c (SREBP-1c), promoting the synthesis of triglycerides and causing lipid metabolism disorders in the liver. The effect of the modified peptide derived from Urechis unicinctus of F2-1 on CYP2E1 activity is shown in FIG. 10. Compared with the blank group: ***P<0.001, **P<0.01, *P<0.05; compared with the model group: ^{###}P<0.001, ^{##}P<0.01, ^{#}P<0.05.

The anti-inflammatory effect of the modified peptide derived from Urechis unicinctus of F2-1 at different concentrations is evaluated based on the levels of IL-1β in HepG2 cells. The effect of the modified peptide derived from Urechis unicinctus of F2-1 on IL-1β activity is shown in FIG. 11. Compared with the blank group: ***P<0.001, **P<0.01, *P<0.05; compared with the model group: ^{###}P<0.001, ^{##}P<0.01, ^{#}P<0.05.

### 1.3 Data Statistics

All data are presented as (x±SD). Statistical analysis is performed using SPSS software. One-way analysis of variance (ANOVA) for independent samples is used to analyze the significance of differences, with P<0.05 considered statistically significant.

### 1.4 Experimental Results

### 1.4.1 ADH Activation Rate Test

Referring to FIG. 1, there is a positive correlation between the activation rate of ADH and the rate of alcohol metabolized in the body. Therefore, the disclosure chose to measure the ADH activation rate to evaluate the ability of the modified peptide derived from Urechis unicinctus to clear alcohol. As shown in FIG. 1, all eight modified peptides derived from Urechis unicinctus listed in Table 1-1 have the ability to activate ADH activity. Among them, F2-1 has the highest ADH activation rate, indicating that F2-1 has the best ability to clear alcohol.

### 1.4.2 Establishment of Alcoholic Damaged HepG2 Cell Model

Referring to FIG. 2, as the concentration of anhydrous ethanol increases, a significant difference in cell viability is observed compared to the blank group (P<0.05). Analysis of FIG. 2 shows that cell viability decreases with increasing concentration of anhydrous ethanol. At 200 mM, there is a significant difference in cell viability due to anhydrous ethanol. At concentrations of 500, 600, and 700 mM, the effect of anhydrous ethanol on cell viability becomes even more pronounced. According to relevant literature, the optimal condition for establishing the alcoholic damaged HepG2 cell model is when cell viability is 50%. Referring to FIG. 2, when the concentration of anhydrous ethanol is 500 mM and the exposure time is 24 hours, the viability of HepG2 cells is 56.83±1.00%, which is statistically significant compared to the blank group. Therefore, the disclosure chose to use 500 mM anhydrous ethanol to treat HepG2 cells for 24 hours to establish the alcoholic damaged HepG2 cell.

1.4.3 Protective Effect of Modified Peptide Derived From Urechis unicinctus of F2-1 at Different Concentrations on Alcoholic Damaged HepG2 Cell

As shown in FIG. 3, compared to the blank group, the cell viability in the model group is 54.69±1.99%, which is significantly lower (P<0.05). Generally, a model is considered successfully established when the damage level is around 50%. Therefore, the alcoholic damaged HepG2 cell model is deemed successful. Compared to the model group, the cell viability in the treatment groups increased with the concentration of modified peptide of F2-1. The low-dose group achieved a cell viability of 80.65±5.67% (P<0.05), the medium-dose group reached 85.39±9.61% (P<0.01), and the high-dose group reached 94.58±4.22% (P<0.001). This indicates that the concentration of modified peptide of F2-1 has a dose-dependent effect on cell viability.

1.4.4 Effects of Modified Peptide Derived From Urechis unicinctus of F2-1 on Biochemical Indicators in HepG2 Cells
1.4.4.1 Effects of Modified Peptide Derived From Urechis unicinctus of F2-1 on ALT and AST Levels in HepG2 Cells

Referring to FIGS. 4 and 5, the effects of the modified peptide derived from Urechis unicinctus of F2-1 on the levels of AST and ALT in HepG2 cells are shown. Compared to the blank group, the levels of AST and ALT in the model group increase significantly (P<0.001), indicating the successful establishment of the alcoholic damaged HepG2 cell model. Compared to the model group, NAC reduces the levels of ALT and AST from 24.07±2.09 units per liter (U/L) and 22.19±0.52 U/L to 7.93±0.14 U/L and 8.26±0.30 U/L, respectively, close to normal levels (P<0.001). In the treatment groups compared to the model group, the low dose has a modest effect, significantly reducing AST to 16.29±0.34 U/L (P<0.001). The medium dose significantly reduces both AST and ALT (P<0.001), with ALT decreasing to 16.28±0.89 U/L and AST to 11.78±0.73 U/L. The high dose has the best effect, significantly reducing AST and ALT (P<0.001) to 8.15±0.81 U/L and 9.45±0.35 U/L, respectively. The results indicate that the modified peptide derived from Urechis unicinctus of F2-1 at different doses can reduce the levels of ALT and AST in HepG2 cells in a dose-dependent manner.

1.4.4.2 Effects of Modified Peptide Derived From Urechis unicinctus of F2-1 on TC and TG Levels in HepG2 Cells

Referring to FIGS. 6 and 7, the effects of the modified peptide derived from Urechis unicinctus of F2-1 on the levels of TC and TG in HepG2 cells are shown. Compared to the blank group, the levels of TC and TG in the model group increase significantly (P<0.01 for TC and P<0.001 for TG), indicating the successful establishment of the alcoholic damaged HepG2 cell model. Compared to the model group, NAC reduces the levels of TC and TG from 0.12±0.02 mmol/L and 0.59±0.02 mmol/L to 0.06±0.02 mmol/L and 0.49±0.00 mmol/L, respectively, close to normal levels (P<0.001). In the treatment groups compared to the model group, the low dose has no significant effect. The medium dose significantly reduces the levels of TC and TG to 0.07±0.01 mmol/L and 0.50±0.01 mmol/L, respectively. The high dose has the best effect, significantly reducing the levels of TC and TG (P<0.001) to 0.06±0.00 mmol/L and 0.46±0.01 mmol/L, respectively. The results indicate that the modified peptide derived from Urechis unicinctus of F2-1 at different doses can reduce the levels of TC and TG in HepG2 cells in a dose-dependent manner.

1.4.4.3 Effects of Modified Peptide Derived From Urechis unicinctus of F2-1 on HDL-C and LDL-C Levels in HepG2 Cells

Referring to FIGS. 8 and 9, the effects of the modified peptide derived from Urechis unicinctus of F2-1 on the levels of HDL-C and LDL-C are shown. Compared to the blank group, the level of HDL-C in the model group decreases significantly (P<0.001), while the level of LDL-C increases significantly (P<0.001), indicating the successful establishment of the alcoholic damaged HepG2 cell model. Compared to the model group, NAC increases the level of HDL-C from 0.04±0.00 mmol/L to 0.12±0.02 mmol/L (P<0.001) and significantly reduces the level of LDL-C from 0.02±0.00 mmol/L to 0.01±0.00 mmol/L (P<0.01). In the treatment groups compared to the model group, the low dose only has a significant effect on HDL-C (P<0.05), increasing its level to 0.08±0.00 mmol/L. The medium dose significantly increases the level of HDL-C (P<0.01) to 0.10±0.02 mmol/L. The high dose has the best effect, increasing the level of HDL-C to 0.12±0.02 mmol/L (P<0.001) and significantly reducing the level of LDL-C (P<0.01) to 0.01±0.01 mmol/L. The results indicate that the modified peptide derived from Urechis unicinctus of F2-1 at different doses can increase the level of HDL-C in HepG2 cells, with the high dose also reducing the level of LDL-C in a dose-dependent manner.

In summary, ALD is often associated with hepatic steatosis caused by lipid metabolism imbalances due to excessive alcohol consumption. The modified peptide of F2-1 can reduce the levels of ALT, AST, TC, TG, and LDL-C in HepG2 cells, indicating its ability to reduce lipid accumulation and subsequent steatosis in hepatocytes, thus providing a protective effect on the liver.

### 1.4.5 Determination of CYP2E1 Content

Referring to FIG. 10, compared to the blank group, the CYP2E1 content in HepG2 cells in the model group increases significantly (P<0.01), indicating the successful establishment of the alcoholic damaged HepG2 cell model. Compared to the model group, the CYP2E1 content in the NAC group decreases significantly from 10.05±0.19 (units per milligram of protein, abbreviated as U/mg prot) to 7.80±0.96 (U/mg prot) (P<0.01). Compared to the model group, the low and medium doses in the treatment groups do not significantly reduce CYP2E1 content, with no statistical significance. The high dose, however, significantly decreases the CYP2E1 content to 7.97±0.78 (U/mg prot) (P<0.01).

The results show that as the concentration of modified peptide derived from Urechis unicinctus increases, the CYP2E1 content in HepG2 cells gradually decreases. This indicates that there is a concentration-dependent relationship between the CYP2E1 content in HepG2 cells and the modified peptide derived from Urechis unicinctus of F2-1.

1.4.6 Effects of Modified Peptide Derived From Urechis unicinctus on the Levels of Inflammatory Cytokines in HepG2 Cell Culture Supernatant

### 1.4.6.1 Determination of IL-1β Content

Referring to FIG. 11, compared to the blank group, the IL-1β content in HepG2 cells in the model group increases significantly (P<0.001), indicating the successful establishment of the alcoholic damaged HepG2 cell model. Compared to the model group, the IL-1β content in the NAC group decreases significantly from 145.94±3.24 picograms per milliliter (pg/mL) to 123.36±1.67 pg/mL (P<0.001). Compared to the model group, the low dose in the treatment group do not significantly reduce IL-1β content, with no statistical significance. The medium dose significantly reduces IL-1β content to 129.62±0.50 pg/mL (P<0.01), and the high dose significantly reduces IL-1β content to 121.33±3.20 pg/mL (P<0.001).

The results show that as the concentration of the modified peptide derived from Urechis unicinctus increases, the IL-1β content in HepG2 cells gradually decreases. This indicates that there is a concentration-dependent relationship between the IL-1β content in HepG2 cells and the modified peptide derived from Urechis unicinctus of F2-1.

### 1.4.6.2 Determination of IL-6 Content

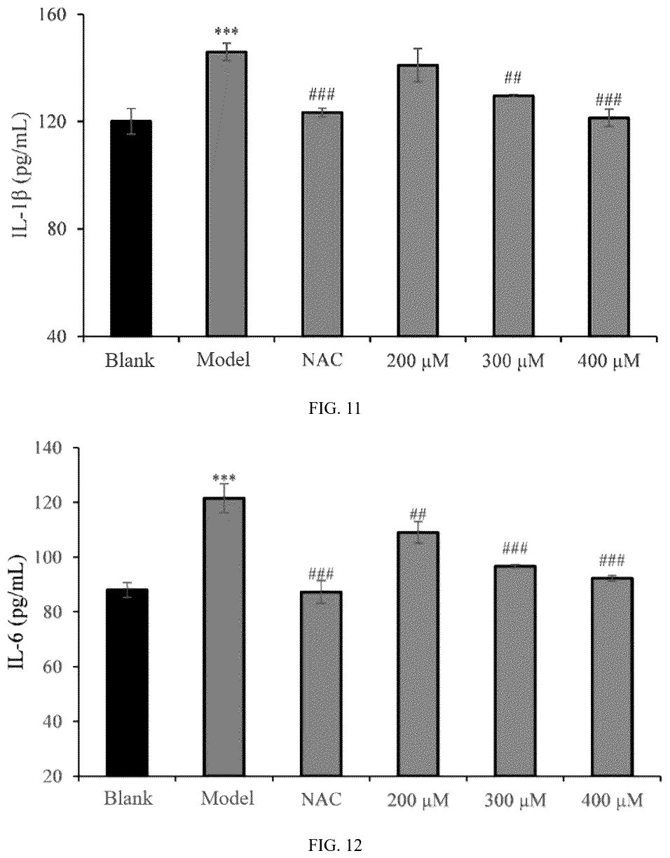

Referring to FIG. 12, compared to the blank group, the IL-6 content in HepG2 cells in the model group increases significantly (P<0.001), indicating the successful establishment of the alcoholic damaged HepG2 cell model. Compared to the model group, the IL-6 content in the NAC group decreases significantly from 121.52±5.30 pg/mL to 87.25±5.30 pg/mL (P<0.001). Compared to the model group, the low dose in the treatment group significantly reduces IL-6 content to 109.01±3.96 pg/mL (P<0.01). The medium and high doses significantly reduce IL-6 content to 96.60±0.57 pg/mL and 92.27±1.02 pg/mL, respectively (P<0.001).

In summary, after treatment with the modified peptide of F2-1, the expression of TNF-α and IL-6 in HepG2 cells is significantly reduced, indicating that the modified peptide of F2-1 may inhibit inflammation by downregulating pro-inflammatory cytokines.

In the embodiment 1, the alcoholic damaged liver model is established by treating healthy HepG2 cells with 500 mM anhydrous ethanol for 24 hours. The in vitro protective effects and mechanisms of the modified peptide derived from Urechis unicinctus of F2-1 against alcoholic liver disease are explored in terms of lipid metabolism and anti-inflammatory activity induced by anhydrous ethanol.

In the disclosure, concentrations of 200 μM, 300 μM, and 400 μM of F2-1 are selected for subsequent experiments to observe its protective effects on HepG2 cells damaged by anhydrous ethanol. The results showed that the modified peptide of F2-1 exhibits good protective effects on the alcoholic damaged liver model in HepG2 cells. In terms of lipid metabolism, different concentrations of the modified peptide of F2-1 could reduce the levels of ALT, AST, TC, TG, and LDL-C in the cells, while increasing the level of HDL-C. Among these, ALT and AST are important indicators for evaluating liver health, and the modified peptide of F2-1 shows a certain degree of improvement in both, indicating that the modified peptide of F2-1 can regulate hepatic fat metabolism, reduce fat accumulation, and provide a certain degree of protection to the liver.

In summary, the modified peptide derived from Urechis unicinctus of F2-1 have certain protective effects against alcoholic liver disease in HepG2 cells caused by anhydrous ethanol. The mechanism of action may be related to the improvement of lipid metabolism and the reduction of inflammatory responses, laying the foundation for subsequent in vivo experiments on the alcohol-damaged liver.

### Embodiment 2

### Protective Effect of Modified Peptide Derived From Urechis unicinctus on Alcohol-Damaged Liver in Mice

### 2.1 Experimental Materials and Instruments

TABLE 2-1
Experimental reagents

| Reagents | Manufacturers |
|---|---|
| Lieber-Decarli Liquid Feed | Nantong Trophic Feed Technology |
| (TP4030C, TP4030D) | Co., Ltd. |
| Anhydrous Ethanol, 95% Ethanol | Sinopharm Chemical Reagent |
| | Co., Ltd. |
| Maltodextrin | Nantong Trophic Feed Technology |
| | Co., Ltd. |
| Triglyceride (TG) Assay Kit | Nanjing Jiancheng Bioengineering |
| | Institute |
| Total Cholesterol (T-CHO) Assay | Nanjing Jiancheng Bioengineering |
| Kit | Institute |
| Silymarin | Tianjin Tasly Pharmaceutical |
| | Group Co., Ltd. |
| Alanine Aminotransferase (ALT) | Nanjing Jiancheng Bioengineering |
| Assay Kit | Institute |
| Aspartate Aminotransferase (AST) | Nanjing Jiancheng Bioengineering |
| Assay Kit | Institute |
| High-Density Lipoprotein | Nanjing Jiancheng Bioengineering |
| Cholesterol (HDL-C) Assay Kit | Institute |
| Low-Density Lipoprotein | Nanjing Jiancheng Bioengineering |
| Cholesterol (LDL-C) Assay Kit | Institute |
| Tumor Necrosis Factor (TNF-α) | Hangzhou Leyi Biotechnology |
| ELISA Assay Kit | Co., Ltd. |
| Interleukin 1β (IL-1β) | Hangzhou Leyi Biotechnology |
| ELISA Assay Kit | Co., Ltd. |
| Interleukin 6 (IL-6) ELISA Assay | Hangzhou Leyi Biotechnology |
| Kit | Co., Ltd. |
| Cytochrome P450 2E1 (CYP2E1) | Hangzhou Leyi Biotechnology |
| ELISA Assay Kit | Co., Ltd. |
| ECL Chemiluminescent Substrate | Shanghai Yease Biopharmaceutical |
| | Technology Co., Ltd. |
| SDS-PAGE Electrophoresis Buffer | Nanjing Jiancheng Bioengineering |
| | Research Institute |
| Western Blot Transfer Buffer | Nanjing Jiancheng Bioengineering |
| | Research Institute |
| TBST | Nanjing Jiancheng Bioengineering |
| | Research Institute |
| Srebp-1c Monoclonal Antibody | Wuhan Three Eagles Biotechnology |
| | Co., Ltd. |
| RNAeasyTM Animal RNA | Beyotime Biotechnology Co., Ltd. |
| Extraction Kit | |
| ABScript III RT Master Mix for | US ABclonal Biological Co., Ltd. |
| qPCR with gDNA Remover | |
| (RK20429) | |
| 2X Universal SYBR Green Fast | US ABclonal Biological Co., Ltd. |
| qPCR Mix (RK21203) | |

### 2.1.1 Experimental Materials

Modified peptide derived from Urechis unicinctus is synthesized by Shanghai Chutai Biotechnology Co., Ltd., with a purity of ≥95%.

### 2.1.2 Experimental Animals

Experimental animals: 48 male C57BL/6 mice of specific pathogen free (SPF) grade, with each mouse weighing between 18 g and 22 g, are purchased from Hangzhou Ziyuan Laboratory Animal Technology Co., Ltd. Production license number: Sheng Chan Xu Ke (SCXK, i.e., production license), (Zhejiang) 2019-0004. All animal experimental procedures complied with the guidelines of the Laboratory Animal Ethics Committee, with a usage license number of Shi Yong Xu Ke (SYXK, i.e., use license) (Zhejiang) 2019-0031.

### 2.1.3 Main Instruments

TABLE 2-2
Main instruments

| Instruments | Manufacturers |
|---|---|
| −80° C. Thermo U/L | Thermo Fisher Scientific (China) |
| Freezer | Co., Ltd. |
| FC3 Gel Documentation System | Bio-Rad Laboratories (Shanghai) |
| | Co., Ltd. |
| GELDOC XR+ Automated Gel | Bio-Rad Laboratories (Shanghai) |
| Documentation System | Co., Ltd. |
| MSA225S Analytical Balance | Sartorius Scientific Instruments |
| | (China) Co., Ltd. |
| Multisjan F Microplate Reader | Bio-Rad Laboratories (Shanghai) |
| | Co., Ltd. |
| PCR Thermal Cycler (T100) | Bio-Rad Laboratories, Inc. (USA) |
| Real-Time PCR System (CFX | Bio-Rad Laboratories, Inc. (USA) |
| Connect) | |
| Ultra-Micro Nucleic Acid | Howo Biotechnology (Shanghai) |
| Detector | Co., Ltd. |
| High-Speed Refrigerated | Dalong Xingchuang Laboratory |
| Centrifuge | Instruments (Beijing) Co., Ltd. |
| DF-101S Constant Temperature | Zhengzhou Ketai Laboratory |
| Heating Magnetic Stirrer | Equipment Co., Ltd. |

### 2.2 Experimental Methods

### 2.2.1 Establishment of the NIAAA Mouse Model and Administration Method

Male C57BL/6 mice (48 in total), weighing 20±2 g, are purchased from Hangzhou Ziyuan Laboratory Animal Technology Co., Ltd. The mice are randomly divided into six groups, with eight mice per group, housed four per cage in a total of 12 cages. The mice are acclimatized in animal facility for seven days at 25±2° C., with a relative humidity of 50±5%, and a 12-hour light/12-hour dark cycle. The experimental groups are as follows: blank group, model group, positive control group, and low-dose, medium-dose, and high-dose groups of modified peptide derived from Urechis unicinctus. The modeling process consisted of four phases. A first phase (administration phase, 10 days): The model group is administered an equivalent volume of distilled water by oral gavage, and the other groups receive the corresponding drug administrations. A second phase (transition phase, 5 days): The mice's diet is gradually transitioned from solid feed to liquid feed. A third phase (modeling phase, 10 days): The blank group continues to receive the Lieber-Decarli liquid diet (alcohol-free), maintaining the same daily caloric intake as the model group. The model group and other treatment groups are fed a liquid diet containing 5% (volume/volume, abbreviated as v/v) alcohol for 10 days. A fourth phase (single gavage phase): On day 11 at 8:00 AM, all groups except the blank group are administered a single oral gavage of 31.5% (v/v) alcohol at a dose of 0.02 mL/g body weight. The mice are euthanized 9 hours later when AST and ALT levels are at their peak. Drug dosages: The positive control group receives silymarin at 200 mg/kg body weight; the low-dose, medium-dose, and high-dose groups receive modified peptide derived from Urechis unicinctus of F2-1 at 50 mg/kg, 100 mg/kg, and 150 mg/kg body weight, respectively. Drug administration continues throughout the entire experiment.

### 2.2.2 Sample Collection and Processing

Liver tissue is homogenized to a 10% liver tissue homogenate

### 2.2.3 Histopathological Observation

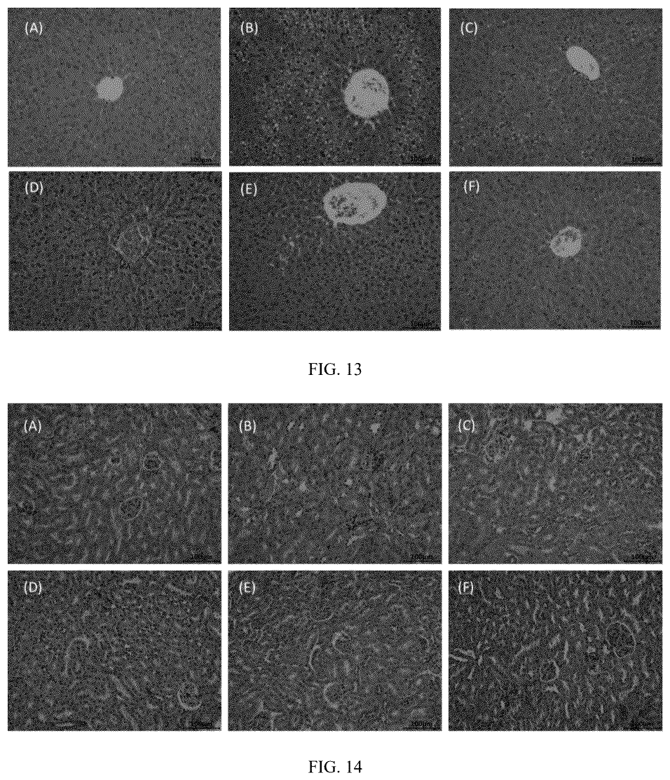

Liver tissue sections and kidney tissue sections are stained with hematoxylin and eosin staining (H&E staining) to observe histopathological changes, with results shown in FIGS. 13 and 14. In FIG. 13: A=blank group; B=model group; C=positive control group; D-low-dose group; E-medium-dose group; F=high-dose group. In FIG. 14: A=blank group; B=model group; C=positive control group; D=low-dose group; E=medium-dose group; F=high-dose group.

### 2.2.4 Determination of Serum Biochemical Indicators in Mice

Same as described in section 1.2.3.2.

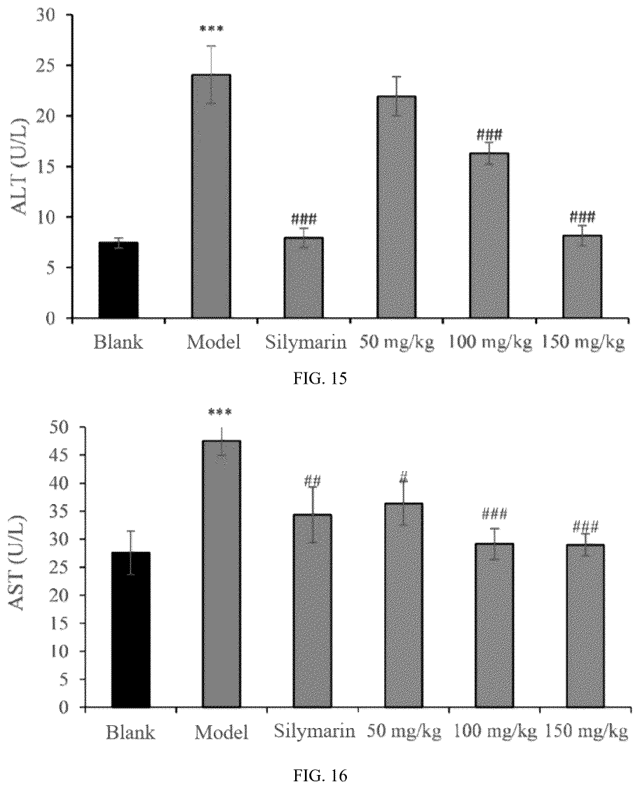

The effect of the modified peptide derived from Urechis unicinctus of F2-1 on ALT content is shown in FIG. 15. Compared with the control group: ***P<0.001, **P<0.01, *P<0.05; compared with the model group: ^{###}P<0.001, ^{##}P<0.01, ^{#}P<0.05.

The effect of the modified peptide derived from Urechis unicinctus of F2-1 on AST content is shown in FIG. 16. Compared with the control group: ***P<0.001, **P<0.01, *P<0.05; compared with the model group: ^{###}P<0.001, ^{##}P<0.01, ^{#}P<0.05.

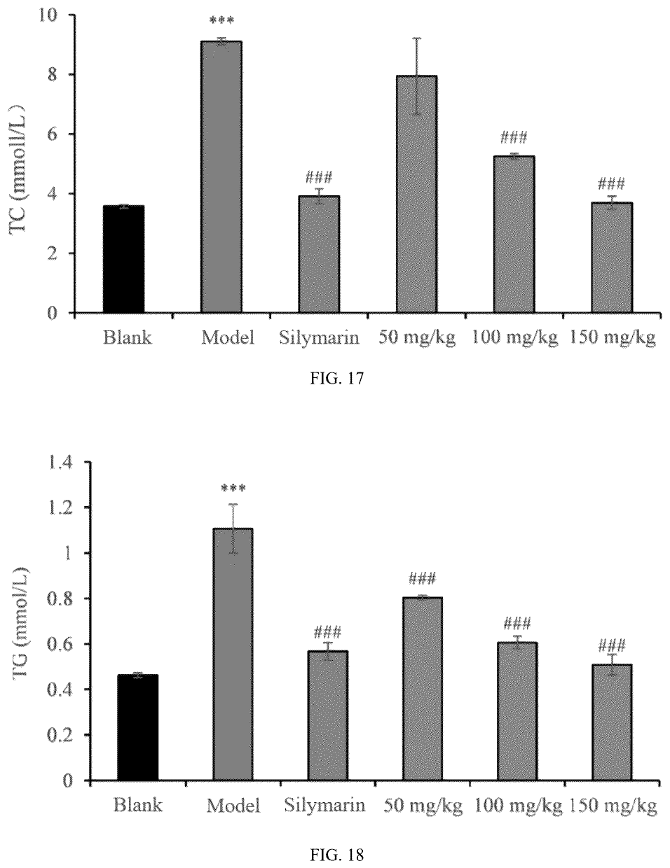

The effect of the modified peptide derived from Urechis unicinctus of F2-1 on TC content is shown in FIG. 17. Compared with the control group: ***P<0.001, **P<0.01, *P<0.05; compared with the model group: ^{###}P<0.001, ^{##}P<0.01, ^{#}P<0.05.

The effect of the modified peptide derived from Urechis unicinctus of F2-1 on TG content is shown in FIG. 18. Compared with the control group: ***P<0.001, **P<0.01, *P<0.05; compared with the model group: ^{###}P<0.001, ^{##}P<0.01, ^{#}P<0.05.

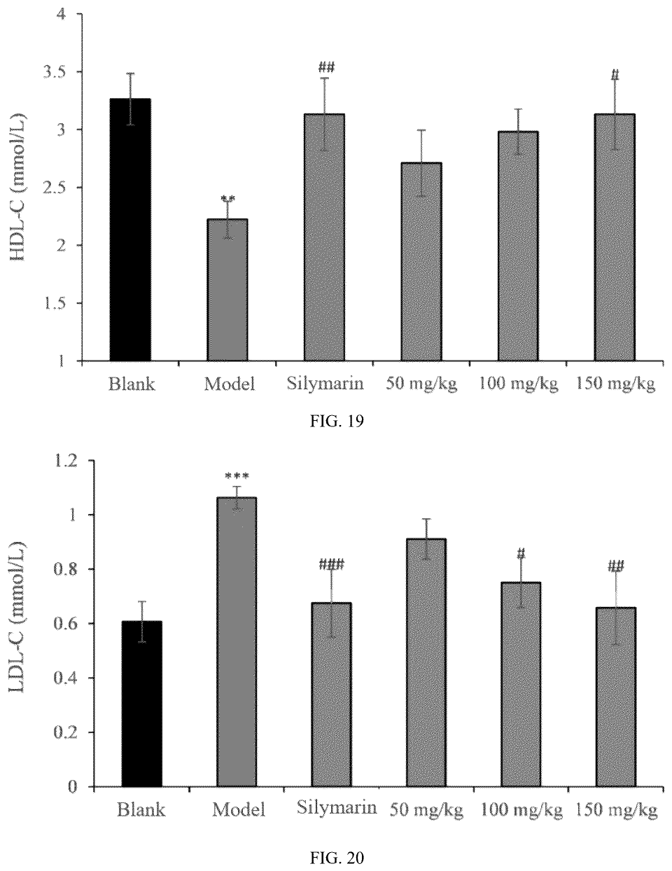

The effect of the modified peptide derived from Urechis unicinctus of F2-1 on HDL-C content is shown in FIG. 19. Compared with the control group: ***P<0.001, **P<0.01, *P<0.05; compared with the model group: ^{###}P<0.001, ^{##}P<0.01, ^{#}P<0.05.

The effect of the modified peptide derived from Urechis unicinctus of F2-1 on LDL-C content is shown in FIG. 20. Compared with the control group: ***P<0.001, **P<0.01, *P<0.05; compared with the model group: ^{###}P<0.001, ^{##}P<0.01, ^{#}P<0.05.

### 2.2.6 Determination of Mouse ELISA Indicators

Same as described in 1.2.3.3

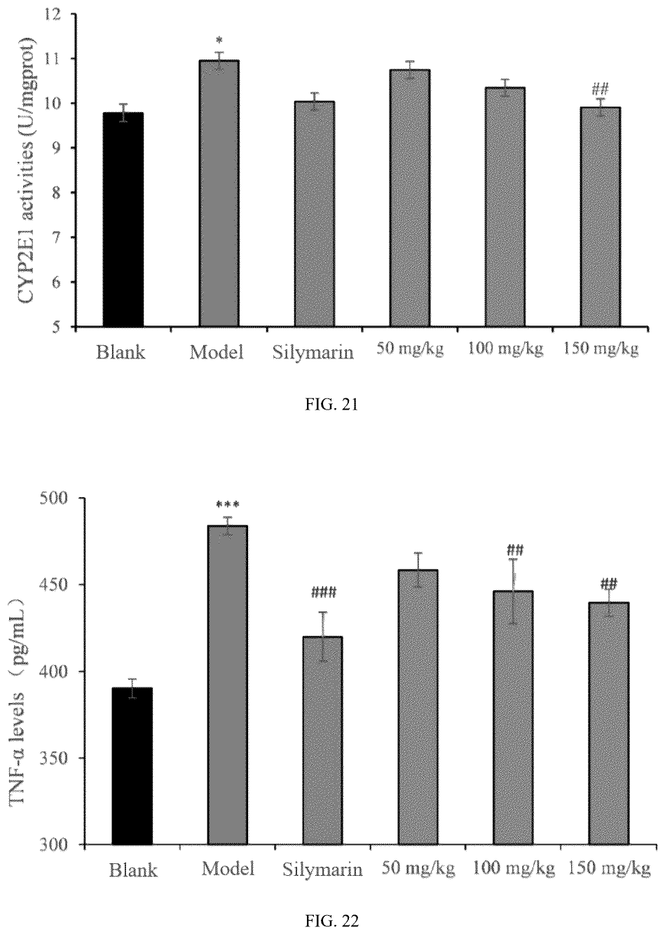

The effect of the modified peptide derived from Urechis unicinctus of F2-1 on CYP2E1 activity is shown in FIG. 21. Compared with the control group: ***P<0.001, **P<0.01, *P<0.05; compared with the model group: ^{###}P<0.001, ^{##}P<0.01, ^{#}P<0.05.

The effect of the modified peptide derived from Urechis unicinctus on TNF-α content is shown in FIG. 22. Compared with the control group: ***P<0.001, **P<0.01, *P<0.05; compared with the model group: ^{###}P<0.001, ^{##}P<0.01, ^{#}P<0.05.

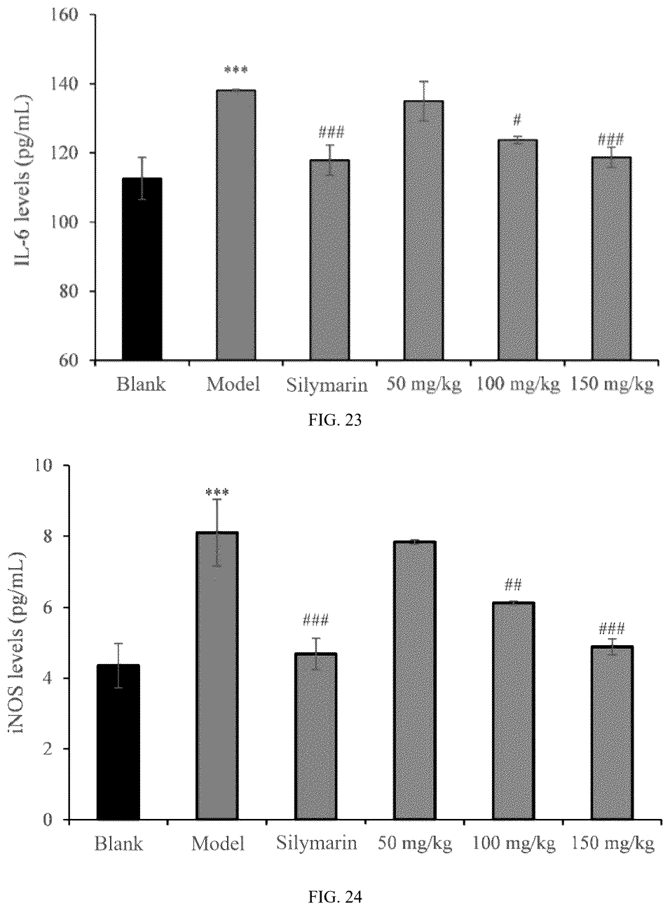

The effect of the modified peptide derived from Urechis unicinctus on IL-6 content is shown in FIG. 23. Compared with the control group: ***P<0.001, **P<0.01, *P<0.05; compared with the model group: ^{###}P<0.001, ^{##}P<0.01, ^{#}P<0.05.

The effect of the modified peptide derived from Urechis unicinctus on inducible nitric oxide synthase (iNOS) content is shown in FIG. 24. Compared with the control group: ***P<0.001, **P<0.01, *P<0.05; compared with the model group: ^{###}P<0.001, ^{##}P<0.01, ^{#}P<0.05.

### 2.3 Statistical Analysis

All data are presented as (x±SD). Statistical analysis is performed using SPSS software. ANOVA for independent samples is used to analyze the significance of differences, with P<0.05 considered statistically significant.

### 2.4 Experimental Results

### 2.4.1 Staining Results

### 2.4.1.1 H&E Staining of Mouse Liver Tissue

Referring to FIG. 13, the H&E-stained liver tissue sections of mice are shown. A portion A of FIG. 13 represents the normal group, which shows neatly arranged hepatocytes with intact nuclei, and the hepatic cords radiate around the central vein. In contrast, a portion B of FIG. 13 represents the model group, which reveals markedly enlarged hepatocytes, disordered hepatic cords, significant inflammatory cell infiltration, and numerous lipid vacuoles under the microscope, confirming the successful establishment of the model. Compared to the model group, the low-dose, medium-dose, and high-dose groups show progressively improved hepatic cord alignment and reduced inflammation. Notably, the high-dose group exhibits well-ordered hepatic cords around the central vein, a marked reduction in lipid vacuoles, and minimal inflammatory cell aggregation, closely resembling the normal state. These findings indicate that the modified peptide derived from Urechis unicinctus of F2-1 at different doses can alleviate alcoholic liver disease in mice to varying extents. Alcohol exposure significantly disrupts hepatic lipid metabolism and contributes to liver damage during ALD progression.

### 2.4.1.2 H&E Staining of Mouse Kidney Tissue

Referring to FIG. 14, the H&E-stained kidney tissue sections of mice are shown. A portion A of FIG. 14 represents the normal group, which shows glomeruli of normal size and shape with clear boundaries and intact brush border structures in the renal tubules. A portion B of FIG. 14 represents the model group, which shows glomerular shrinkage, blurred boundaries, and disordered renal tubule structures compared to the blank group, indicating the successful establishment of the alcoholic damaged liver model. Compared to the model group, all dose groups (low, medium, and high) demonstrate improvement, with the high-dose group showing nearly normal glomerular size and shape, clear boundaries, and intact brush border structures in the renal tubules. This suggests that the modified peptide derived from Urechis unicinctus of F2-1 at various doses can reduce alcoholic damaged kidney in mice in a dose-dependent manner.

2.4.2 Effects of Modified Peptide Derived From Urechis unicinctus of F2-1 on Mouse Serum Indicators
2.4.2.1 Effects of Modified Peptide Derived From Urechis unicinctus of F2-1 on ALT and AST Levels in Mouse Serum

Referring to FIGS. 15 and 16, the modified peptide derived from Urechis unicinctus of F2-1 significantly reduces ALT and AST levels in the mouse serum. Compared with the blank group, the model group shows a significant increase in ALT and AST (P<0.001), confirming the successful establishment of the NIAAA model. This suggests that excessive alcohol consumption damages the liver. Compared with the model group, silymarin reduces ALT and AST from 24.07±2.85 U/L and 47.57±2.63 U/L to 7.93±0.90 U/L and 34.32±4.97 U/L, respectively. The low-dose group only reduces AST (P<0.05), to 36.35±3.91 U/L. The medium dose significantly reduces ALT and AST (P<0.001), to 16.28 =1.08 U/L and 29.19±2.75 U/L, respectively. The high dose shows the best effect, significantly reducing ALT and AST (P<0.001), to 8.15±0.99 U/L and 28.97±1.94 U/L, respectively. The results show that the modified peptide derived from Urechis unicinctus of F2-1 at different doses can reduce ALT and AST levels in mouse serum with alcoholic liver disease, with a dose-dependent effect and partial alleviation of alcoholic liver disease.

2.4.2.2 Effects of Modified Peptide Derived From Urechis unicinctus of F2-1 on TC and TG Levels in Mouse Serum

As shown in FIGS. 17 and 18, the effects of modified peptides derived from Urechis unicinctus on the TC content and the TG content are shown. Excessive alcohol consumption leads to excessive accumulation of TC and TG in hepatocytes, disrupting liver metabolic function. Compared with the blank group, the model group shows a significant increase in TC and TG (P<0.001), confirming the successful establishment of the NIAAA model. Compared with the model group, silymarin significantly reduces TC and TG to near-normal levels (P<0.001), from 9.10±0.12 mmol/L and 1.10±0.10 mmol/L to 3.91±0.25 mmol/L and 0.57±0.04 mmol/L, respectively. Compared with the model group, the low dose has no significant effect on TC but significantly reduces TG to 0.80±0.01 mmol/L (P<0.001). The medium dose significantly reduces TC and TG (P<0.001), to 5.25±0.09 mmol/L and 0.61±0.03 mmol/L, respectively. The high dose shows the best effect, significantly reducing TC and TG (P<0.001), to 3.69±0.21 mmol/L and 0.51±0.04 mmol/L, respectively. The results show that the modified peptide derived from Urechis unicinctus of F2-1 at different doses can reduce TC and TG levels in mouse serum with alcoholic liver disease, with a dose-dependent effect and partial alleviation of alcoholic liver disease.

2.4.2.3 Effects of Modified Peptide Derived From Urechis unicinctus of F2-1 on HDL-C and LDL-C Levels in Mouse Serum

Referring to FIGS. 19 and 20, the effects of modified peptide derived from Urechis unicinctus of F2-1 on HDL-C and LDL-C levels are shown. Compared with the blank group, the model group shows a significant decrease in HDL-C and a significant increase in LDL-C (P<0.001), confirming the successful establishment of the NIAAA model, that is to say, excessive alcohol consumption causes liver damage in mice. Compared with the model group, silymarin increases HDL-C from 2.22±0.16 mmol/L to 3.13±0.31 mmol/L (P<0.01) and significantly reduces LDL-C from 1.06±0.04 mmol/L to 0.67±0.12 mmol/L (P<0.001). Compared with the model group, the low dose has no significant effect on HDL-C and LDL-C, which is not statistically significant. The medium dose reduces LDL-C to 0.75 =0.09 mmol/L (P<0.05). The high dose shows the best effect, increasing HDL-C to 3.13±0.30 mmol/L (P<0.05) and significantly reducing LDL-C to 0.65±0.13 mmol/L (P<0.01). The results show that the modified peptide derived from Urechis unicinctus of F2-1 at different doses can increase HDL-C levels and reduce LDL-C levels in mouse serum with alcoholic liver disease, with a dose-dependent effect and partial alleviation of alcoholic liver disease.

In summary, ALD is often accompanied by hepatic steatosis caused by lipid metabolism imbalances due to excessive alcohol consumption. The modified peptide of F2-1 can reduce ALT, AST, TC, TG, and LDL-C levels and increase HDL-C levels in NIAAA mice, indicating that the modified peptide of F2-1 reduces lipid accumulation and steatosis in hepatocytes and has a protective effect on the liver.

### 2.4.3 Determination of CYP2E1 Activity

Referring to FIG. 21, the modified peptide derived from Urechis unicinctus of F2-1 significantly affects CYP2E1 activity. Compared to the blank group, the model group shows a slight increase in CYP2E1 activity (P<0.05), confirming the successful establishment of the NIAAA model and indicating liver damage due to alcohol. Compared to the model group, silymarin and low/medium doses of the modified peptide of F2-1 have no significant effect. However, the high dose of the modified peptide of F2-1 significantly reduces CYP2E1 activity from 10.94±0.11 U/mgprot to 9.90±0.60 U/mgprot (P<0.05). Chronic alcohol consumption activates hepatic CYP2E1, inducing oxidative stress and subsequent tissue inflammation and damage.

The results indicate that the modified peptide derived from Urechis unicinctus of F2-1 can increase hepatic CYP2E1 levels in mice with alcoholic liver disease, with a dose-dependent effect and partial alleviation of alcoholic liver disease.

2.4.4 Effects of Modified Peptide Derived From Urechis unicinctus of F2-1 on Inflammatory Cytokines in Mouse Liver Tissue

### 2.4.4.1 Determination of TNF-α Content

Referring to FIG. 22, the modified peptide derived from Urechis unicinctus of F2-1 significantly reduces TNF-α content in mouse liver tissue. Compared to the blank group, the model group shows a significant increase in TNF-α (P<0.001), confirming the successful establishment of the NIAAA model, that is to say, excessive alcohol consumption causes liver damage in mice. Compared to the model group, silymarin reduces TNF-α from 483.77±5.13 pg/mL to 420±14.09 pg/mL (P<0.001). The low-dose group has no significant effect, which is not statistically significant, while the medium and high doses significantly reduce TNF-α to 446.087±18.44 pg/mL and 439.56±7.74 pg/mL, respectively (P<0.01).

The results show that the modified peptide derived from Urechis unicinctus of F2-1 at different doses can reduce TNF-α levels in mouse liver tissue with alcoholic liver disease, with a dose-dependent effect and partial alleviation of alcoholic liver disease.

### 2.4.4.2 Determination of IL-6 Content

Referring to FIG. 23, the modified peptide derived from Urechis unicinctus of F2-1 significantly reduces IL-6 content in mouse liver tissue. Compared to the blank group, the model group shows a significant increase in IL-6 (P<0.001), confirming the successful establishment of the NIAAA model, that is to say, excessive alcohol consumption causes liver damage in mice. Compared to the model group, silymarin reduces IL-6 from 138.08±0.29 pg/mL to 117.88±4.41 pg/mL (P<0.001). The low-dose group has no significant effect, which is not statistically significant, while the medium dose reduces IL-6 to 123.70±1.06 pg/mL (P<0.05), and the high dose reduces IL-6 to 118.71±2.90 pg/mL (P<0.001).

The results show that the modified peptide derived from Urechis unicinctus of F2-1 at different doses can reduce IL-6 levels in mouse liver tissue with alcoholic liver disease, with a dose-dependent effect and partial alleviation of alcoholic liver disease.

2.4.4.3 Determination of iNOS Content

Referring to FIG. 24, the modified peptide derived from Urechis unicinctus of F2-1 significantly reduces iNOS content in mouse liver tissue. Compared to the blank group, the model group shows a significant increase in iNOS (P<0.001), confirming the successful establishment of the NIAAA model, that is to say, excessive alcohol consumption causes liver damage in mice. Compared to the model group, silymarin reduces iNOS from 8.10±0.93 pg/mL to 4.68±0.44 pg/mL (P<0.001). The low-dose group has no significant effect, which is not statistically significant, while the medium dose reduces iNOS to 6.12±0.05 pg/mL (P<0.01), and the high dose reduces iNOS to 4.88±0.23 pg/mL (P<0.001). The results show that the modified peptide derived from Urechis unicinctus of F2-1 at different doses can reduce iNOS levels in mouse liver tissue with alcoholic liver disease, with a dose-dependent effect and partial alleviation of alcoholic liver disease.

In summary, after treatment with the modified peptide of F2-1, the expression of TNF-α, IL-6, and iNOS in liver cells of NIAAA mice is significantly reduced, indicating that the modified peptide of F2-1 may inhibit inflammation by downregulating pro-inflammatory cytokines.

In the embodiment 2, male ICR mice (20±2 g) are used. After a week of adaptive growth, they are gavaged with different doses of the modified peptide derived from Urechis unicinctus of F2-1 for 10 days. Then, they are fed with adaptive feed for 5 days with continued medication. An alcoholic damaged liver model is established using liquid feed containing anhydrous ethanol for 10 days. The protective effects and mechanisms of the modified peptide derived from Urechis unicinctus of F2-1 on alcoholic damaged liver in mice are explored in terms of lipid regulation and anti-inflammation. The doses of the modified peptide of F2-1 are set at 50 mg/kg, 100 mg/kg, and 150 mg/kg to observe its protective effects.

In the alcoholic damaged liver model, the results show that compared to the normal group, the model group exhibits increased hepatic lipid vacuoles, disordered hepatic cords, and inflammatory infiltration in H&E staining, confirming the successful model establishment. The drug-administered group shows improvement, indicating that the modified peptide of F2-1 could enhance lipid metabolism and reduce lipid accumulation.

In terms of lipid metabolism, different concentrations of the modified peptide of F2-1 reduce ALT, AST, TC, TG, and LDL-C levels in the body and increase HDL-C levels. ALT and AST are key indicators of liver health, and the modified peptide of F2-1 shows certain improvement effects, suggesting the modified peptide of F2-1 can regulate hepatic fat metabolism, reduce fat deposition, and provide liver protection.

In terms of anti-inflammatory effects, different concentrations of the modified peptide of F2-1 reduce TNF-α, IL-6, and iNOS levels. The modified peptide of F2-1 can inhibit inflammation by reducing inflammatory markers and regulating anti-inflammatory genes, protecting against alcoholic liver disease.

In summary, in the alcoholic damaged liver model, the modified peptide derived from Urechis unicinctus of F2-1 protects mice by improving lipid metabolism and reducing inflammation. It significantly protects against alcoholic liver disease and inhibits pro-inflammatory cytokines in serum. As a potential liver-protective bioactive peptide, the modified peptide derived from Urechis unicinctus of F2-1 shows great application potential.

What is claimed is:

1. An application method of a modified peptide derived from Urechis unicinctus, comprising:
preparing a drug for treating alcoholic liver disease using the modified peptide derived from Urechis unicinctus;
wherein the modified peptide derived from Urechis unicinctus is at least one selected from the group consisting of:
F2-1 with the amino acid sequence of SEQ ID NO: 1: IHVKF,
F2-2 with the amino acid sequence of SEQ ID NO: 2: VHFKI,
F4-1 with the amino acid sequence of SEQ ID NO: 3: GAWKP,
F4-2 with the amino acid sequence of SEQ ID NO: 4: AGWKP,
F5-1 with the amino acid sequence of SEQ ID NO: 5: GTLKP,
F5-2 with the amino acid sequence of SEQ ID NO: 6: GTPKL,
F7-1 with the amino acid sequence of SEQ ID NO: 7: IIVRM, and
F7-2 with the amino acid sequence of SEQ ID NO: 8: IRMVI.

2. The application method of the modified peptide derived from Urechis unicinctus as claimed in claim 1, wherein the modified peptide derived from Urechis unicinctus is capable of activating an alcohol dehydrogenase (ADH) enzyme, thereby enhancing a degradation efficiency of alcohol.

3. The application method of the modified peptide derived from Urechis unicinctus as claimed in claim 1, wherein the modified peptide derived from Urechis unicinctus is capable of increasing a survival rate of damaged liver cells.

4. The application method of the modified peptide derived from Urechis unicinctus as claimed in claim 1, wherein the modified peptide derived from Urechis unicinctus is capable of reducing lipid accumulation and hepatic steatosis in alcoholic liver cells.

5. The application method of the modified peptide derived from Urechis unicinctus as claimed in claim 4, wherein the modified peptide derived from Urechis unicinctus is capable of regulating a level of at least one selected from the group consisting of alanine transaminase (ALT), aspartate aminotransferase (AST), total cholesterol (TC), triglycerides (TG), low-density lipoprotein cholesterol (LDL-C), and high-density lipoprotein cholesterol (HDL-C) in the alcoholic liver cells.

6. The application method of the modified peptide derived from Urechis unicinctus as claimed in claim 1, wherein the modified peptide derived from Urechis unicinctus is capable of suppressing a pro-inflammatory cytokine in serum and reducing inflammatory responses in alcoholic liver cells.

7. The application method of the modified peptide derived from Urechis unicinctus as claimed in claim 6, wherein the pro-inflammatory cytokine is at least one selected from the group consisting of cytochrome P450 2E1 (CYP2E1), interleukin-1B (IL-1B), and interleukin-6 (IL-6).

8. A drug for treating alcoholic liver damage, comprising the modified peptide derived from Urechis unicinctus as claimed in claim 1.


SEQUENCE LISTING (8 sequences)

SEQ ID NO: 1 - protein, 5 aa, Urechis unicinctus
IHVKF

SEQ ID NO: 2 - protein, 5 aa, Urechis unicinctus
VHFKI

SEQ ID NO: 3 - protein, 5 aa, Urechis unicinctus
GAWKP

SEQ ID NO: 4 - protein, 5 aa, Urechis unicinctus
AGWKP

SEQ ID NO: 5 - protein, 5 aa, Urechis unicinctus
GTLKP

SEQ ID NO: 6 - protein, 5 aa, Urechis unicinctus
GTPKL

SEQ ID NO: 7 - protein, 5 aa, Urechis unicinctus
IIVRM

SEQ ID NO: 8 - protein, 5 aa, Urechis unicinctus
IRMVI